(19) United States
(12) Patent Application Publication
(10) Pub. No.: US 2004/0151757 A1
(43) Pub. Date: Aug. 5, 2004

(54) Use of medium-chain triglycerides (MCT) for optimisation as to nutrition physiology of the fatty acid spectrum in a dietary foodstuff for diabetics

(75) Inventor: Horst Heirler, Woerthsee (DE)

Correspondence Address: FOLEY AND LARDNER, SUITE 500, 3000 K STREET NW, WASHINGTON, DC, 20007, US

(73) Assignee: Horst Heirler Projekte Ernahrung * Medizin * Okologie

(21) Appl. No.: 10/717,990

(22) Filed: Nov. 21, 2003

(30) Foreign Application Priority Data
Nov. 22, 2002 (DE) 102 54 584.7-41

(51) Int. Cl. A61K 31/225; A61K 47/00

(52) U.S. Cl. 424/439; 514/547

(57) ABSTRACT

A method for supplementing the diet of a subject with diabetes mellitus comprising administering to the subject medium-chain triglycerides (MTC) for optimization as to food physiology of the fatty acid spectrum in a dietary foodstuff. The use of MCT and a dietary foodstuff containing these MTC for particular medicinal purposes for supplementary monitored nutrition/dietary treatment of adults and teenagers with diabetes mellitus is described.

### BACKGROUND OF THE INVENTION

The present invention relates to the use of MCT or a dietary foodstuff containing these for particular medicinal purposes for supplementary monitored nutrition/dietary treatment of adults and teenagers with diabetes mellitus.

Diabetes mellitus relates to a group of diseases associated with a chronic regulatory disorder of the metabolism. Characteristic for it is a chronic hyperglycosemia resulting from a defect in the insulin secretion or in the effect of the insulin. The two main types are the so-called type-1 diabetes which is due to an absolute lack of insulin and the so-called type-2 diabetes characterised in a reduced secretion and/or a reduced sensitivity to insulin of the peripheral tissue. The frequency of both types is at a ratio of about 1:9.

Clinically, it is important that, with diabetes mellitus, chronic hyperglycosemia leads to vessel changes in the form of the macro- and microangiopathy and to nerve damage. These resulting diseases are caused by long-term organ impairments, above all of the blood vessels, the heart, the kidneys, the eyes and the nerves. The irreversible changes of long-lived molecules in cells, in particular in cell nuclei, which are due to high glucose concentrations, are of central importance in the development of diabetic macro- and microangiopathy. The non-enzymatic formation of stable glycosylation products (“advanced glycosylation products”) are the basis of the mechanism of such impairments. They depend on the extent and the duration of the glucose accumulation and take effect in the context and in interaction with other aggressive factors such as free radicals and/or lipid peroxidation products.

The chronic regulation impairment relates to not only the carbon hydrate metabolism but also to the fat, protein and electrolyte metabolism. When the kidney threshold of glucose is exceeded, the hyperglycosemia leads to glucosuria, a lack of glucose in the muscle and fat tissue and to an increase of the gluconeogenesis. The impairment of the fat metabolism results in a limited synthesis and storage of neutral fats. As a consequence, the mobilisation of depot fats is stimulated and the metabolic fat conversion is increased. The increased formation of acetacetic acid and beta-hydroxy butanoic acid triggers a ketose and increased oxidative stress occurs. Since the formation of lipoproteins also intensifies, their degradation, however, is delayed, dyslipoproteinemia due to peroxidatively changed lipoproteins appears. The impaired protein metabolism is characterised by an inhibition of the protein synthesis and an increased protein degradation. The amino acids released increasingly enter the gluconeogenesis and the energy metabolism. In addition, in the case of lack of insulin, the electrolyte metabolism is impaired. The consequences are cellular potassium loss and secondary disruptions of the water, sodium and acid-base balance.

Whereas meanwhile the feared acute metabolism failures (coma diabeticum, coma hypoglycaemicum) can be controlled easily, the above chronic consecutive diseases caused by diabetes mellitus and later damage have taken a central role in the health problems of diabetics.

The medicinal treatment regarding nutrition represents an integral column of the diabetes therapy. Objects of this therapy are: (a) the normalisation of the glucose level in the blood to reduce or even prevent the risk of complications, in particular consecutive diseases; (b) a lipid and lipoprotein profile which reduces the risk of macroangiopathia and (c) the achievement of blood pressure values which reduce the risk of vessel diseases. The metabolic conditions therefore can and should be achieved by a change of lifestyle, in particular a modified nutrition. They are suitable for the prevention and treatment of adiposity, hyperlipoprotein anaemia, cardiovascular diseases, hypertension and kidney diseases.

For the diabetic, an optimal metabolism control is without doubt the best prevention and therapy. It protects against macro- and microangiopathia and reduces the risk thereof and the same applies to other complications and consecutive diseases. In this context, the nutrition with insulin resistance and type-2 diabetes plays a role, in particular within the context of the “metabolic syndrome”. This is accompanied by insulin resistance, adiposity, cardiovascular diseases, hypertonia and dyslipoproteinemia. About 80% of type-2 diabetics are in fact overweight. Abdominal obesity, physical inactivity, dyslipoproteinemia and defective glucose tolerance are the main causes of a manifestation of diabetes. Thus, the metabolic syndrome is the classic risk of the development of early arteriosclerosis.

The therapeutic and preventive approach regarding nutrition for the delay and—even better—for the prevention of the late complications mentioned provide the regulation and normalisation of the lipid metabolism, as is always the case with the prevention of arteriosclerosis. In this context, the following risk factors are preferably to be assumed: (a) overweight, in particular abdominal adiposity, as a consequence of too high a supply of food energy and fat, (b) consumption of long-chain saturated fatty acids in food and trans-unsaturated fatty acids, (c) furthermore, a ratio of alkane, mono and polyalkyl fatty acids which is unfavourable physiologically and for hemodynamics, (d) a relation of omega-6 to omega-3 nutritional fatty acids >5:1, (e) an insufficient provision of long-chain omega-3 fatty acids (C20:5=eicosapentaen acid and C22:6=docosahexaen acid), (f) an insufficient prevention of the peroxidation of lipids rich in polyalkene of the food and, consequently, in vivo the LDL (low density lipoproteins), too, due to an insufficient exogenous provision of anti-oxidative protective substances, especially of vitamin E and C and (g) an insufficient supply of vegetable sterols.

Thus, it is the object of nutritional therapy in diabetes to reduce or even prevent the above risks of arterosclerotic vessel diseases by means of normoglycemia. According to general agreement and also according to the recommendations for the nutrition of diabetics published by the Diabetes and Nutrition Group (DNSG) of the European Association for the Study of Diabetes (EAS) and the “Ausschuss Ernädhrung der Deutschen Diabetes-Gesellschaft” (DDG) [Nutrition Committee of the German Diabetes Association] 2000 and in a position paper published by the American Diabetes Association (ADA) 2002, this means to aspire to a body-mass-index of 18.5 to 25 kg/m² by reducing the uptake of nutritional fat energy to 25 to 35% of the total energy, supplying 60 to 70% of the nutritional energy % by carbon hydrates and monosaturated fatty acids having cis-configuration and to divide the distribution of the nutritional fat energy as follows: (1) saturated, specifically long-chain fatty acids+transunsaturated fatty acids: <8% (ADA)<7%), (2) cis-monounsaturated fatty acids (mainly oleic acid): 10 to 20%, (3) polyunsaturated fatty acids (polyalkene fatty acids, PUFA): ≦10%, (4) a ratio of omega-6:omega-3 fatty acids ≦5:1, (5) long-chain polyalkene fatty acids (fish oil fatty acids; C20:5=eicosapentaen acid; C22:6=docosahexaen acid): ≧0.2% and (6) promotion and purpose-orientated increase of the uptake of anti-oxidative food ingredients (food anti-oxidants). Moreover, it is known that the consumption of monounsaturated fatty acids in amounts of ≧15% of nutritional energy reduces an increased lipid serum level.

Such recommendation for diabetics largely corresponds to the one for the average population as expressed by the nutrition associations of Germany, Austria and Switzerland in their D-A-CH Reference Values of 2000. The DGG holds a somewhat different view in so far as it sees the energy uptake from nutritional fats in a more liberal way, since it considers the quality of the fatty acids supplied to be more crucial and strongly focuses on the low glycemic index of foods. Indeed, in the average of the population (according to the Nutrition Report 2000 of the German Association for Nutrition), food fats still constitute about 36% of the food energy and carbon hydrates constitute only 44 to 46%. Saturated fatty acids provide nearly 15% of the food energy, monounsaturated ones about 13.5% and polyunsaturated ones 5.7%. The ratio of omega-6 to omega-3 fatty acids is 8:1. The maximum amount of fish oil fatty acids formed from the amount of alpha-linolenic acid (C18:3 omega-3) absorbed on average is 0.15 g/day. If one assumes the amount supplied when eating fish to be 0.1 g/day, there is a daily supply of 0.25 g eicosapentaen+docosahexaen acid. This constitutes only about 0.1% of the food energy supply and not, as recommended, >0.2% of energy. In addition, a considerable part of the (German) population (according to the results of a representative survey by the Berlin Robert-Koch-Institute published in 2002) does not fulfil the D-A-CH reference values for vitamins. For the anti-oxidative vitamins E and C, it is e.g. about 60% and 24%.

Thus, the promotion and purposeful increase of the uptake of anti-oxidative food ingredients considered necessary for diabetics has no good starting position. In view of this general nutrition situation, it is essential when changing the food fat consumption, above all, to reduce the uptake of saturated fatty acids, to exclude, if possible, the uptake of trans-fatty acids and to increase the uptake of unsaturated ones. This should be done in favour of the omega-3 fatty acids and at the expense of the omega-6 fatty acids. Furthermore, the necessity becomes obvious to significantly increase the consumption of food anti-oxidants.

In dietary practice, the uncontrolled change of the consumption of nutritional fat rather takes place by omitting spreading and cooking fats as well as cooking and salad oils than by reducing the consumption of foods of animal origin and of backed, deep-fried, fried products and products with fat-containing coatings with a high proportion of saturated fatty acids in the so-called hidden fats. Therefore and since there are neither margarines and other spreading fats nor cooking fats, salad, cooking and frying oils in a make-up which comes up to the above recommendations, there is always the risk for diabetics that during maintaining or reducing of their fat consumption, the result is a fatty acid pattern in the food which does not correspond to the one recommended and which, as a consequence, counteracts a regulation and normalisation of the lipid metabolism within the meaning of arteriosclerosis prevention-in the organism via the fatty acid composition of the lipoprotein and membrane phospholipids. Although margarines with and without fat reduction (80 and 60% fat) are known which, apart from oleic acid (C 18:1 omega-9) and the essential fatty acids linoleic acid (C18:2 omega-6) and alpha-linolenic acid (C18:3 omega-3), also exhibit eicosapentaen acid (C20:5 omega-3) and docosahexaen acid (C22:6 omgega-3) at a physiologically appropriate ratio. However, due to the use of hardened fats as solid components, they have the disadvantage that they contain long-chain and unsaturated trans-fatty acids. Furthermore, the amount and concentration of the vitamins added to the fats suffice, at best, for the oxidation protection of the unsaturated fatty acids contained in them as food components.

### SUMMARY OF THE INVENTION

Thus, the technical problem underlying the present invention is to provide a dietary foodstuff for patients with diabetes mellitus which does not exhibit these disadvantages so that pathological changes of the fat metabolism can be treated by dietary therapy and, thus, helps to regulate and normalise the fat metabolism and, in that way prevents the consecutive diseases which reduce the quality of life and, in the end, are life-threatening, preferably macro- and microangiopathiae.

This problem has been solved by providing the embodiments characterised in the claims. Surprisingly, it was found that medium-chain triglycerides (MCT) are suitable as hard fats for the production of spreading fats (margarines) and cooking fats which meet the dietary requirements from a medicinal point of view stated above and that the suitability is due to not only a technological view but also to a dietary point of view with regard to physiology and medicine. As a spreading and cooking fat and as an oil, the composition and/or the dietary foodstuff according to the invention represents a composition and a ratio of the fatty acids, for the first time, in a way which has to be requested for the dietary regime of diabetics as a principle.

### DETAILED DESCRIPTION OF THE INVENTION

In brief, the inventory achievement, above all, is that (a) for the first time, medium-chain triglycerides (MCT) are used for monitored supplementary dietary treatment of diabetics for the regulation and for optimising of the metabolism situation and (b) these, preferably in combination with unsaturated fatty acids, in particular the long-chain omega-3 fatty acids, are used for the particular metabolic requirements of diabetics as monitored supplementary diet in the form of spreading fats and/or cooking oils.

The use of a specific percentage of such medium-chain triglycerides, preferably containing nearly exclusively caprylic acid (C8:0) and/or capric acid (C10:0), called MCT below, has, in combination with oleic acid (C18:1) and, in particular, with long-chain (LCT) omega-3 fatty acids from fish oils, the following crucial advantages: (a) by avoiding the lymph passage via the portal vein, MCT reach the liver directly and are oxidised therein; (b) MCT are not stored in fat tissue, (c) MCT have a lower calorific value of 8.3 kcal/g fat than long-chain fatty acids which have a calorific value of 9.3 kcal/g fat.

For that reason alone, a reduction of the uptake of fat energy is achieved and necessary weight reductions are favoured. The use of a relatively high part of oleic acid (C18:1 omega-9) already improves the flexibility and the deformability of the phospholipid-containing membranes of blood cells and, thus, of haemodynamics and reduces the risk of fatal clotting processes at the same time. The use of a highly purified concentrate of omega-3 fish oil fatty acids (C20:5, C22:6) outweighs the disadvantage of the omega-3 fatty acid alpha linolenic acid (C18:3) introduced with plant oils, i.e. in an endogenous way, usually only up to 2 to 7% and, in exceptional cases, up to 10% maximum, being converted into the LCT omega-3 fatty acid (C20:5 and C22:6) and, thus, not fulfilling the necessary supply of these.

Due to the change of the fatty acid spectrum achieved according to the invention, there is a comprehensive influence of the metabolic situation in diabetics. In this context, the change of the synthesis of eicosanoids in the organism plays an essential role. Due to the increased formation of eicosanoids from eisosapentaen acid (C20:5 omega-3) instead from arachidonic acid (C20:4 omega-6; this is consumed with both foods of animal origin and in the human organism formed from linoleic acid [C18:2 omega-6]), there is a reduction of the risk potential of arterosclerosis. The risk potential in this context results from an increased aggregation and adhesion of the thrombocytes, an increased vasoconstriction and increased inflammation reactions.

Thus, the present invention relates to the use of medium-chain triglycerides (MCT) and/or a composition containing medium-chain triglycerides for the dietary treatment of people with diabetes mellitus.

The term “medium-chain triglyceride” used herein relates to triglycerides with a nearly exclusive content of octane acid (caprylic acid; C8:0) and/or decane acid (capric acid; C10:0) according to the systemic and trivial name in the chemical nomenclature.

The person skilled in the art knows coconut and palm seed fat, preferably, as sources for these medium-chain glycerides.

The exclusive use of MCT fats as dietary component is limited. Therefore, the supply of MCT in the form of MCT-containing foods, e.g. margarines, so that there are many possibilities for the diabetic to consume them. Since, so far, plant oils and oil mixtures, too, are not disposable for cooking and frying purposes and for the preparation of salads and other dishes with the fat acid spectrum desirable for diabetics, it is advantageous to transfer the principle of the solution of the problem according to the invention to salad and cooking and frying oil, too.

In a preferred embodiment, the dietary foodstuff according to the invention therefore contains comparable fat acid compositions and ratios as fat/oil mixture such as the fat phases of margarines and other spreading fats with the above explained claim with regard to dietary physiology and medicinal aspect of a particular suitability for the diet of diabetics. In this case, only the long-chain fish oil fatty acids are not considered, since they are sensitive to heat and particularly susceptible to oxidation, which could have a disadvantageous effect regarding the taste of the dishes prepared therewith.

In this embodiment, however, MCT are also an integral part of cooking oils for the preparation in the kitchen of salads, soups and stews, sauces, dressings, dips, ketchups, chutneys and other sauces for seasoning, mayonnaises, remoulade sauces and all other dishes which are made consumable or ready for consumption by using plant, cooking, frying, deep-frying and barbecue oils and fats and the like.

According to the definition and purpose of the guideline 1999/21/EC dated 25 Mar. 1999 (and their transfer into the German law in the form of the Diet Regulation in the version of the notification of 25.08.1988, latest amendment by Art. 1 Tenth Amendment of Diet Regulation dated 21.12.2001), the products according to the invention and described by way of example below are incomplete foodstuff with regard to diet with a standard formulation specifically adjusted for a specific disease or disorder, which are not suitable for the use as single source of food. Rather, their particular dietary purpose is the monitored supplement of an individual basic diet which normalise the glycemic control and, thus, is to prevent and/or minimise complications and secondary consequences. In this context, apart from the relation of the main nutrients carbon hydrate, fats and proteins, particular attention has to be paid to the quality of the food fats and the ratio of their fatty acids to and amongst each other. This is easily achieved by the purposeful consumption of the spreading fats (margarines) and frying, cooking and salad oils according to the invention.

In a preferred use according to the invention, in the fat phase, the composition contains (a) 10 to 30% medium-chain triglycerides, (b) monoene acid(s) (monounsaturated fatty acids), preferably oleic acid (C18:1), preferably 20 to 60%, and/or (c) diene acid(s) (double unsaturated fatty acids) such as linoleic acid (C18:2), preferably 10 to 35%, and/or (d) triene acid(s) (triple unsaturated fatty acids) such as alpha linolenic acid, preferably 3 to 10%.

The main sources for oleic acids are olive, rapeseed and canola oil. Preferably, linoleic acid is derived from sunflower and rape oil. The alpha linolenic acid is primarily gained from rape oil; however, linseed oil also represents a suitable source thereof.

In order to prevent an insufficient supply of long-chain polyalkene acids (multi-unsaturated fatty acids; C20:5=eicosapentaen acid and C22:6=docosahexaen acid), in a particularly preferred use of the invention, the composition in the fat phase further contains eicosapentaen acid and/or docosahexaen acid, preferably from sea animal fats and, especially, from mixtures of highly-purified (refined) fish oils, wherein values of 0.5 to 2% eicosapentaen acid and/or docosahexaen acid in form of triglycerides are preferred.

In a further preferred use of the invention, the content of saturated long-chain (>12 C-atoms) fatty acids (alkane fatty acids) in the composition is 6% at the most. These can be derived from plant fats such as olive, rape and sunflower oil or coco fat and/or cow milk butter.

In an even more preferred use of the invention, the composition in the fat phase is the following:

| | |
|---|---|
| (a) medium-chain triglycerides | 10 to 30%; |
| (b) saturated long-chain fatty acids | 0.5 to 6%; |
| (c) oleic acid | 20 to 60%; |
| (d) linoleic acid | 10 to 35%; |
| (e) alpha-linolenic acid | 3 to 10%; and |
| (f) eicosapentaen and/or docosahexaen acid | 0.5 to 2%. |

Preferably, the fat phase of the composition for the use of the invention contains, apart from the components described above, in addition as emulsifiers, mono- and diglycerides from edible fatty acids (MDG such as e.g. E 471), but no phosphatides such as lecithin, which occur as natural emulsifiers, for example as side products during purification. In addition, there are fat-soluble vitamins, preferably the vitamins A, D, E and/or vitamin C in the form of ascorbyl palmitate, β-carotin, butter and/or—with regard to highly-unsaturated fatty acid—suitable spicy flavourings such as e.g. rosemary extracts.

With regard to the fat soluble vitamins listed above, the following concentrations in the fat phase of 100 g of the emulsified end product are particularly preferred: 0.0002 to 0.002 g retinyl palmitate (vitamin A), 1 to 5 μg (40 to 200 I. U.) vitamin D₃ (Cholecalciferol), 0.06 to 0.6 g ascorbyl palmitate, 0.02 to 0.2 g RRR-α-tocopheryl acetate (natural vitamin E).

As to the basic diet it has now surprisingly been found out that with the dietary foodstuff of the invention as supplementary monitored diet, the glycemic control can also be achieved and the regulation of the metabolism which is disturbed in many ways in the case of diabetes mellitus can be improved by adding specific B vitamins as elementary metabolic co-enzymes to the aqueous phase of spreading fats (margarines).

In a preferred embodiment, therefore, e.g. a dietary spreading fat (margarine) contains, apart from the vitamins belonging to the metabolic elementary B vitamins, also vitamin B₆, folic acid and vitamin B₁₂, since these three B vitamins play an important role in the homocysteine metabolism. The hyperhomocysteinemia is a further autonomous risk factor for vascular diseases as complications of diabetes mellitus, which can be dealt with a good supply of these three vitamins.

In the most preferred use, the vitamins are present in the compositions in the aqueous phase in the following amounts (and preferred compounds): 0.01 to 0.25 g vitamin C (as sodium ascorbate); 0.0005 to 0.005 g vitamin B₁ (as thiamine mononitrate); 0.0006 to 0.006 g vitamin B₂ (as riboflavin-5′-sodium); 0.0007 to 0.007 g vitamin B₆ (as pyridoxine hydrochloride); 0.0015 to 0.015 mg B₁₂ (as cyanocobalamin); 0.007 to 0.07 g niacin (as nicotinamide); 0.0002 to 0.002 g folic acid (as pteroyl monoglutamate).

In an alternative embodiment, instead of a highly purified fish oil concentrate, salts of the trace elements zinc, chrome and/or manganese are added to guarantee a genuine supply with eicosapentaen and docosahexaen acid of the aqueous phase.

As co-factors and activators, zinc, chrome and manganese contribute to the regulation and normalisation of the intermediary metabolism of diabetics in an effective manner. Furthermore, it is known that in most cases diabetics lack zinc and that there is a connection between zinc, the glucose metabolism and insulin. There is a similar connection between glucose metabolism and the supply of chrome.

Accordingly, in an alternative particularly preferred use of the invention, the composition additionally contains zinc, chrome and/or manganese in the aqueous phase. The corresponding salts of the trace elements may be any chemical compounds listed in the guideline 2001/15/EG of 15.02.2001 and, accordingly, in Enclosure 2, List A of § 7 paragraph 1 sentence 1 No. 1, paragraph 2 of the German Diet Regulation (Diät-VO) in the version of 21.12.2001, which are admissible as additives for special dietary purposes within the framework of dietary plans for a special diet and for special purposes with regard to food physiology and diet. Preferably, the compounds of the invention are substances which have a high aqueous solubility and which do not have a pro-oxidative effect during use. Particularly suitable concentrations are 0.00225 to 0.015 g zinc; 0.03 to 0.1 mg chrome and 0.002 to 0.005 g manganese in the aqueous phase of 35 to 40 g of the end products exhibiting a fat phase of 60 to 65 g and, thus, a total mass of 100 g.

In the most preferred embodiment of the use according to the invention, the composition (and the dietary foodstuff) for supplementary monitored diet/dietary treatment of diabetes mellitus in the form of a spreading fat (margarine) has a fat content of 65% and per 100 g the following composition: energy content: 558 kcal; saturated fatty acids: 13 g, 10 g of which are medium-chained triglycerides (MCT); monounsaturated fatty acids (oleic acid): 29 g; multiple unsaturated fatty acids: 19 g, of which 14 g are linoleic acid (omega-6), 4.25 g are alpha-linolenic acid (omega-3), 0.75 g are eicosapentaen and docosahexaen acid (omega-3); energy-%-quotient omega-6 to omega-3 fatty acids=2.9:1; vitamin D₃: 150 I. U.; vitamin A: 690 μg; RRR-α-tocopherol (vitamin E): 100 mg; vitamin C: 200 mg; vitamin B₁: 4 mg; vitamin B₂: 4.5 mg; vitamin B₆: 5 mg; niacin: 53 mg; folic acid: 1.3 mg; vitamin B₁₂: 10 μg; β-carotin: 8 μg; sodium <0.05 g; free of trans-fatty acids from hardened fats.

### EXAMPLE

Composition and Production of a Margarine for the Supplementary Monitored Diet/Dietary Treatment of Adults and Teenagers with Diabetes Mellitus

Due to its main components, the composition (and the dietary foodstuff) for the use according to the invention is preferably put onto the market in the form of margarines and/or cooking oils.

As margarine, the dietary foodstuff according to the invention preferably contains either 20 or 35% water in the aqueous phase and, accordingly, either 80 or 65% fat in the fat phase. As cooking oil, the dietary foodstuff according to the invention preferably contains about 99.5% fat in total; this oil is a mixture of vegetable oils and MCT fat.

Examples of recipes are given in the Tables 1 and 2 below.

TABLE 1
Recipes for fats for diabetics

| Margarine with 80% fat | | | margarine with 65% fat | | | oil | | |
|---|---|---|---|---|---|---|---|---|
| rape oil | 35.0 | g | rape oil | 45.0 | g | rape oil | 69.43 | g |
| MCT fat | 18.0 | g | MCT fat | 10.0 | g | MCT oil | 15.0 | g |
| olive oil | 13.0 | g | | | | | | |
| sunflower oil | 10.0 | g | sunflower | 7.0 | g | sunflower | 15.0 | g |
| | | | oil | | | oil | | |
| fish oil concentrate^{a} | 3.0 | g | fish oil concentrate^{a} | 3.0 | g | | | |
| retinyl palmitate | 0.001 | g | retinyl palmitate | 0.0011 | g | | | |
| RRR-α-tocopheryl | 0.10 | g | RRR-α-tocopheryl | 0.10 | g | RRR-α- | 0.10 | g |
| acetate | | | acetate | | | tocopheryl | | |
| | | | | | | acetate | | |
| Ascorbyl palmitate | 0.470 | g | ascorbyl palmitate | 0.470 | g | ascorbyl | 0.470 | g |
| | | | | | | palmitate | | |
| mono- and di- | 0.331 | g | mono- and di- | 0.6 | g | | | |
| glycerides of edible | | | glycerides of edible | | | | | |
| fatty acids | | | fatty acids | | | | | |
| cholecalciferol | 150 | I. U. | Cholecalciferol | 150 | I. U. | | | |
| β-carotin^{b} | 0.008 | mg | β-carotin^{b} | 0.0065 | mg | | | |
| butter flavouring | 0.090 | g | butter flavouring | 0.001 | g | | | |
| oil-soluble | | | oil-soluble | | | | | |
| Cooking salt^{d} | 0.10 | g | cooking salt | 0.10 | g | | | |
| citric acid | 0.005 | g | citric acid | 0.042 | g | | | |

| | | | | |
|---|---|---|---|---|
| butter flavouring | 0.005 | g | butter flavouring | q. s. |
| water-soluble | | | water-soluble^{c} | |

^{a}Highly-purified fish oil concentrate stabilised with RRR-α-tocopheryl and ascorbyl palmitate which contains at least 30% long-chain omega-3-polyalkene fatty acids.
^{b}preferably with a 30% suspension at the most
^{c}if necessary with regard to taste;
^{d}copper and iron content below 1 part per million (ppm)

TABLE 2

| Margarine with 80% fat | | | Margarine with 65% fat | | |
|---|---|---|---|---|---|
| aqueous | 19.89 | g with | aqueous | 33.6819 | g with |
| solution of B | | | solution of B | | |
| vitamins with | | | vitamins with | | |
| thiamine | 220 | mg | thiamine | 130 | mg |
| mononitrate/L | | | mononitrate/L | | |
| riboflavin-5′- | | | riboflavin-5′- | | |
| phosphate- | | | phosphate- | | |
| sodium/L | | | sodium/L | | |
| pyridoxine | 310 | mg | pyridoxine | 185 | mg |
| hydrochloride/L | | | hydrochloride/L | | |
| nicotin amide/L | 305 | mg | nicotin amide/L | 180 | mg |
| folic acid/L | 2665 | mg | folic acid/L | 1575 | mg |
| cyanocobalamin/ | 65 | mg | cyanocobalamin/ | 40 | mg |
| L | 0.5 | mg | L | 0.3 | mg |

Production

(a) Fat Phase

The MCT fat is melted, optionally with further natural fats mainly containing medium-chain triglycerides, wherein the temperature must not be higher than 60° C. Then, the vegetable oils are added at the same temperature. The fish oil concentrate and the other fat-soluble ingredients are added to the liquid mixture no sooner than immediately before homogenisation.

(b) Aqueous Phase

All the water-soluble ingredients are dissolved in de-ionised and de-aired water, and the solution is pasteurised.

Then, the aqueous phase is slowly added to the fat phase at 40 to 50° C., and a homogenous emulsion is formed in a cutter block rotating with high speed. Crystallisation and kneading to a product which is easy to spread take place in a manner typical for the production of margarine, i.e. with a “votator” consisting of A- and B-units (scraped surface heat exchanger and resting tube).

Packing is carried out at 15° C., and the product is stored at a cool place, optionally frozen (freezer). In general, keeping time at 4° C. is 3 months, under particularly good circumstances (fried, no access of atmospheric oxygen), also 6 months and longer.

1. A method for supplementing the diet of a subject with diabetes mellitus comprising administering to the subject medium-chain triglycerides or a composition comprising medium-chain triglycerides in an amount sufficient to regulate and normalize fat metabolism in the subject.

2. The method according to claim 1, wherein the composition in the fat phase comprises:
(a) 10 to 30% medium-chain triglycerides;
(b) at least one monounsaturated fatty acid, and/or
(c) linoleic acid; and/or
(d) α-linolenic acid.

3. The method according to claim 2, wherein the monounsaturated fatty acid is oleic acid.

4. The method according to claim 3, wherein the composition comprises 20 to 60% oleic acid as monounsaturated trigylceride.

5. The method according to claim 2, wherein the composition comprises 10 to 35% linoleic acid as double-unsaturated triglyceride.

6. The method according to claim 2, wherein the composition comprises 3 to 10% α-linolenic acid as triple-unsaturated triglyceride.

7. The method according to claim 2, wherein the composition in the fat phase further comprises eicosapentaen acid and/or docosahexaen acid as mutiple-unsaturated triglycerides.

8. The method according to claim 7, wherein the composition comprises 0.5 to 2% eicosapentaen acid and/or docosahexaen acid.

9. The method according to claim 1, wherein the composition further comprises saturated long-chain triglycerides of 6% at the most.

10. The method according to claim 1, wherein the composition in the fat phase comprises:

| | |
|---|---|
| (a) medium-chain triglycerides | 10 to 30%; |
| (b) saturated long-chain triglycerides | 0.5 to 6%; |
| (c) oleic acid | 20 to 60%; |
| (d) linoleic acid | 10 to 35%; |
| (e) alpha-linolenic acid | 3 to 10%; and |
| (f) eicosapentaen acid and/or docosahexaen acid | 0.5 to 2%. |

11. The method according to claim 2, wherein the fat phase further comprises as emulsifiers, mono- and diglycerides of edible fatty acids, fat-soluble vitamins, #-carotene, butter flavourings and/or flavourings which are suitably spicy and anti-oxidative with regard to the highly unsaturated fatty acids.

12. The method according to claim 11, wherein the fat-soluble vitamins are vitamins A, D, E and/or vitamin C in the form of ascorbyl palmitate.

13. The method according to claim 12, wherein the fat phase of the composition comprises 0.0002 to 0.002 g retinyl palmitate and/or 1 to 5 μg (40-200 I. U.) vitamin D₃ and/or 0.02 to 0.2 g natural vitamin E in the form of RRR-α-tocopheryl acetate and/or 0.06 to 0.6 g ascorbyl palmitate.

14. The method according to claim 2, wherein (a) the fat phase of the composition comprises about 80% and the aqueous phase is about 20% or (b) the fat phase of the composition is about 60 to 65% and the aqueous phase is 35 to 40%.

15. The method according to claim 14, wherein the aqueous phase comprises the vitamins B₆, B₁₂ and/or folic acid.

16. The method according to claim 15, wherein the aqueous phase further comprises the vitamins C, B₁, B₂ and/or niacin.

17. The method according to claim 16, wherein the composition comprises 0.01 to 0.25 g vitamin C and/or 0.0005 to 0.005 g vitamin B₁ and/or 0.0006 mg to 0.006 g vitamin B₂ and/or 0.0007 to 0.007 g vitamin B₆ and/or 0.0015 to 0.015 mg vitamin B₁₂ and/or 0.007 to 0.070 g niacin (nicotine amide) and/or 0.0002 to 0.002 g folic acid.

18. The method according to claim 14, wherein the aqueous phase of the composition contains zinc, chrome and/or manganese.

19. The method according to claim 18, wherein the composition per 100 g comprises 0.00225 to 0.015 g zinc and/or 0.03 mg to 0.1 mg chrome and/or 0.002 to 0.005 g manganese.
